(19) United States
(12) Patent Application Publication
(10) Pub. No.: US 2017/0172459 A1
(43) Pub. Date: Jun. 22, 2017

(54) Methods and Devices for Monitoring Breathing and Sound Data of a Patient

(71) Applicant: Pneumonics, Inc., Durham, NC (US)

(72) Inventors: Kenneth Bernstein, Durham, NC (US); Bryce Hill, Butte, MT (US)

(21) Appl. No.: 15/380,096

(22) Filed: Dec. 15, 2016

Related U.S. Application Data
(60) Provisional application No. 62/269,569, filed on Dec. 18, 2015.

(51) Int. Cl. A61B 5/08; A61B 5/113; A61B 5/00; A61B 7/00

(52) U.S. Cl. CPC A61B 5/0816; A61B 7/003; A61B 5/1135; A61B 5/7278; A61B 5/6822; A61B 5/0015; A61B 5/7282; A61B 5/746; A61B 5/6823; A61B 2562/0219

(57) ABSTRACT

Devices and methods for monitoring respiratory activity of a patient. The system includes a first sensor for sensing the acoustics of the patient during breathing and a second sensor for sensing chest wall motion of the patient during breathing. Specific aspects of the patient's breathing can be determined based on the sensor readings. This may include whether the patient is experiencing regular breathing, slow breathing (hypopnea), fast breathing (tachypnea), no breathing (apnea), or obstructed breathing. The methods and devices may further be configured to trigger an alarm in predetermined situations.

### REFERENCE TO RELATED APPLICATIONS

This application claims the benefit of U.S. Provisional Application No. 62/269,569, filed Dec. 18, 2015, the contents of which are hereby incorporated by reference in their entirety.

### BACKGROUND

Various devices and methods are used to monitor the breathing of a patient. The monitoring may determine whether the patient is experiencing regular breathing, slow breathing (hypopnea), fast breathing (tachypnea), no breathing (apnea), or obstructed breathing.

Respiratory complications account for roughly 10% of hospital adverse events (Department of Health and Human Services. Office of Inspector General (2010). Adverse Events in Hospitals: National Incidence Among Medicare Beneficiaries). Multiple factors can contribute to complications of the respiratory system. These include infection or diseases of the airway tracts, infection or diseases of the lung tissue, complete or partial obstruction of the airway tracts and altered control of breathing by the brain.

Respiration is measured clinically as minute ventilation, or the volume of air a person breathes over one minute. Diseases or conditions that impact lung volume, respiratory rate or both will effect minute ventilation. Respiratory depression is defined as minute ventilation that is below the normal threshold. Of particular concern is the widespread use of opioids, and their effect on the brain's ability to control normal breathing. Opioids have a profound negative effect on minute ventilation by effecting both lung volume and respiratory rate. Opioid induced respiratory depression is a serious and potentially life-threatening side effect of opioid painkillers.

The rate of opioid induced respiratory depression is as high as 60% (Overdyk, F. J., et al. (2007). Continuous Oximetry/Capnography Monitoring Reveals Frequent Desaturation and Bradypnea During Patient-Controlled Analgesia. Anesthesia & Analgesia, 105(2), pp. 412-418). Medical experts agree that opioid related injury or death is avoidable with adequate respiratory monitoring. Despite the availability of respiratory rate monitors, injury or death from opioid induced respiratory depression is common. Approximately 40% of in-hospital emergency calls are for opioid related respiratory depression (Fecho, K., Jackson, F., Smith, F., & Overdyk, F. J. (2009). In-Hospital Resuscitation: Opioids and Other Factors Influencing Survival. Therapeutics and Clinical Risk Management, 5, pp. 961-968).

Respiratory monitoring of a person who is breathing spontaneously without the assistance of a ventilator is difficult task. There are several respiratory monitoring technologies on the market. Pulse oximetry measures oxygen levels by comparing red light and infra red light absorption ratios in the blood. Impedance pneumography estimates minute ventilation by measuring voltage potential changes in the chest during respiration. Capnography measures respiratory rate by analyzing breath samples for the presence of carbon dioxide. Each of these respiratory monitors is beset by unacceptably high false alarm rates.

Acoustic analysis of tracheal breath sound is a clinically useful tool to detect respiratory rate and upper airway obstruction. Tracheal breath sounds have been extensively classified, and cover a frequency range from 100 Hz to 1,500 Hz (Gavriely, N., Palti, Y., & Alroy, G. (1981). Spectral Characteristics of Normal Breath Sounds. Journal of Applied Physiology, Respiratory, Environmental and Exercise Physiology, 50(2), pp. 307-314). The power of tracheal sounds exhibit peaks and troughs that are co-related with airway diameter. For example, upper airway obstruction that occurs during sleep apnea produces a characteristic snoring sound, with an increase in mean spectral power across the entire frequency range (Sarkar, M., Madabhavi, I., Niranjan, N., & Dogra M. (2015). Auscultation of the Respiratory System. Annals of Thoracic Medicine, 10(3), pp. 158-168).

Observation of breathing is a necessary component of a physical exam. Muscle activity of the rib cage and diaphragm contribute to the movement of the thorax during respiration. Analysis of thoracic motion can help detect abnormal breathing patterns. For example, chest wall motion that occurs without simultaneous audible breath sounds is a clear sign of an upper airway obstruction.

An accelerometer is one embodiment of a tool for assessing chest and abdominal wall motion during respiration. Accelerometer applications in medical devices have been described as early as 1983. Researchers placed an accelerometer on the tip of an implantable pacemaker lead that sensed patient physical activity and adjusted heart rate as necessary (Humen, D. P., Anderson, K., Brumwell, D., Huntley, S., & Klein, G. J. (1983). A Pacemaker Which Automatically Increases its Rate with Physical Activity. In K. Steinbach, Cardiac Pacing (pp. 259-264). Dresden, Germany: Steinkopff).

Existing methodologies for detecting sounds include an audio sensor that detects the sounds during patient breathing. However, these sensors often detect additional noises in the patient's environment. These may include persons talking in the vicinity of the patient, equipment noises, and general background noise. It is often difficult or impossible to filter out the additional noises and leave just the breathing sounds. When the additional noises are included in the data that is analyzed, the noises often result in inaccurate monitoring results.

### SUMMARY

The present application is directed to methods and devices for monitoring patient breathing. The patient breathing is detected based motion and sound that is taken concurrently while the patient is breathing. The phases of the breath cycles of the patient breathing can be determined and the used to analyze the breathing.

One embodiment is directed to a method of monitoring breathing of a patient. The method includes: receiving chest wall motion and breath sounds that is sensed concurrently while the patient is breathing; determining components of the breath cycle of the patient based on the chest wall motion, with each of the breath cycles comprising an inspiratory phase, an inspiratory pause phase, an expiratory phase, and an expiratory pause phase; determining a beginning time and an ending time for each of the phases based on the chest wall motion; and determining at least one of an acoustic respiratory rate and a motion respiratory rate of the patient based on the sound during one or more of the inspiratory phase, the expiratory phase, and the expiratory pause phase as determined through the chest wall motion.

The method may also include establishing a baseline for the chest wall motion and determining for each breath cycle that the inspiratory phase begins when the motion data passes through the baseline in a positive direction and the expiratory phase ends when the motion data passes through the baseline in a negative direction.

The method may also include determining that the expiratory pause phase occurs between an end of the expiratory phase in a first breath cycle and a start of the inspiratory phase in a second breath cycle. This method may also include determining that the chest wall motion remains below the baseline during the entirety of the expiratory pause phase.

The method may include that establishing the baseline for the chest wall motion includes calculating an average inclination of the sensor over a period of time.

The method may include determining a motion respiratory rate based on the chest wall motion for the breath cycles that occur over a period of time using the chest wall motion only for the breath cycles that have a maximum chest wall motion that is above a predetermined threshold.

The method may include: calculating a noise floor threshold based on the breathing sound during the expiratory pause phase of a plurality of the breath cycles; applying the noise floor threshold to the breathing sound; calculating a number of edge crossings at which the breathing sound crosses over the noise floor threshold; and determining an acoustic respiratory rate based on the number of edge crossings that occur within a predetermined time period.

Another embodiment is directed to a system for monitoring breathing of a patient. The system includes: a first sensor to sense chest wall motion while the patient is breathing; a second sensor to concurrently sense sound and sound that is taken while the patient is breathing; a processing circuit configured to: determine components of the breath cycle of the patient based on the chest wall motion with each of the breath cycles comprising an inspiratory phase, an inspiratory pause phase, an expiratory phase, and an expiratory pause phase; determine a beginning time and an ending time for each of the phases based on the chest wall motion; and determine at least one of an acoustic respiratory rate and a motion respiratory rate of the patient based on the sound during one or more of the inspiratory phase, the expiratory phase, and the expiratory pause phase as determined through the chest wall motion.

The processing circuit may be further configured to establish a baseline for the chest wall motion and determine for each breath cycle that the inspiratory phase begins when the motion data passes through the baseline in a positive direction and the expiratory phase ends when the motion data passes through the baseline in a negative direction.

The processing circuit may be further configured to determine that the expiratory pause phase occurs between an end of the expiratory phase in a first breath cycle and a start of the inspiratory phase in a second breath cycle.

The processing circuit may be configured to determine that the chest wall motion remains below the baseline during the entirety of the expiratory pause phase.

The system may include that establishing the baseline for the chest wall motion includes the processing circuit calculating an average inclination of the sensor over a period of time.

The processing circuit may be configured to determine a motion respiratory rate based on the chest wall motion for the breath cycles that occur over a period of time using the chest wall motion only for the breath cycles that have a maximum chest wall motion that is above a predetermined threshold.

The processing circuit may be configured to calculate a noise floor threshold based on the breathing sound during the expiratory pause phase of a plurality of the breath cycles; apply the noise floor threshold to the breathing sound; calculate a number of edge crossings at which the breathing sound crosses over the noise floor threshold; and determine an acoustic respiratory rate based on the number of edge crossings that occur within a predetermined time period.

Another embodiment is directed to a system for monitoring breathing of a patient. The system includes a processing circuit configured to: receive chest wall motion and sound that are concurrently sensed while the patient is breathing; determine components of the breath cycle of the patient based on the chest wall motion with each of the breath cycles comprising an inspiratory phase, an inspiratory pause phase, an expiratory phase, and an expiratory pause phase; determine a beginning time and an ending time for each of the phases based on the chest wall motion; and determine at least one of an acoustic respiratory rate and a motion respiratory rate of the patient breathing based on the sound during one or more of the inspiratory phase, the expiratory phase, and the expiratory pause phase as determined through the chest wall motion.

Another embodiment is directed to a method of monitoring breathing of a patient. The method includes: sensing chest wall motion comprising an inclination of a sensor over time relative to two or more orthogonal axes; while sensing the chest wall motion, also sensing breathing sounds of the patient; calculating breath cycles of the patient based just on the patient motion with each of the breath cycles comprising an inspiratory phase, an inspiratory pause phase, an expiratory phase, and an expiratory pause phase, each of the phases having a start and an end; and using the breathing sound data from at least one of the inspiratory phase, the expiratory phase, and the expiratory pause phase as determined through the motion data and determining at least one of an acoustic respiratory rate and a motion respiratory rate.

The method may also include establishing a baseline for the chest wall motion and determining that the inspiratory phase begins for each breath cycle when the motion data passes through the baseline in a positive direction.

The method may also include determining that the expiratory phase ends when the motion data passes through the baseline in a negative direction.

The method may also include determining that the expiratory pause phase occurs between an end of the expiratory phase in a first breath cycle and a start of the inspiratory phase in a second breath cycle.

The method may also include determining that the motion data remains below the baseline during the entirety of the expiratory pause phase.

The method may also include that establishing the baseline for the chest wall motion comprises calculating an average angle over a period of time.

The method may also include determining whether a maximum chest wall motion during the inspiratory phase and the expiratory phase is above an upper motion threshold.

The method may also include determining a motion respiratory rate based on the chest wall motion for the breath cycles that occur over a period of time using the chest wall motion only for the breath cycles that have a maximum chest wall motion that is above a predetermined threshold.

Another embodiment is directed to a system to monitor breathing of a patient. The system includes: a first sensor to sense chest wall motion including an inclination of a sensor over time relative to two or more orthogonal axes; a second sensor to concurrently sense breathing sounds of the patient; a processing circuit configured to: calculate breath cycles of the patient based just on the patient motion with each of the breath cycles comprising an inspiratory phase, an inspiratory pause phase, an expiratory phase, and an expiratory pause phase, each of the phases having a start and an end; use the breathing sound data from at least one of the inspiratory phase, the expiratory phase, and the expiratory pause phase as determined through the motion data and determine at least one of an acoustic respiratory rate and a motion respiratory rate.

The processing circuit may be configured to establish a baseline for the chest wall motion and determine that the inspiratory phase begins for each breath cycle when the motion data passes through the baseline in a positive direction.

The processing circuit may be configured to determine that the expiratory phase ends when the motion data passes through the baseline in a negative direction.

The processing circuit may be configured to determine that the expiratory pause phase occurs between an end of the expiratory phase in a first breath cycle and a start of the inspiratory phase in a second breath cycle.

The processing circuit may be configured to determine that the motion data remains below the baseline during the entirety of the expiratory pause phase.

The processing circuit may be configured that establishing the baseline for the chest wall motion includes the processing circuit calculating an average angle over a period of time.

The processing circuit may be configured to determine whether a maximum chest wall motion during the inspiratory phase and the expiratory phase is above an upper motion threshold.

The processing circuit may be configured to determine a motion respiratory rate based on the chest wall motion for the breath cycles that occur over a period of time using the chest wall motion only for the breath cycles that have a maximum chest wall motion that is above a predetermined threshold.

Another embodiment is directed to a system to monitor breathing of a patient. The system includes a processing circuit configured to: receive chest wall motion and breathing sounds that are sensed concurrently while a patient is breathing; calculate breath cycles of the patient based just on the patient motion with each of the breath cycles comprising an inspiratory phase, an inspiratory pause phase, an expiratory phase, and an expiratory pause phase, each of the phases having a start and an end; use the breathing sound data from at least one of the inspiratory phase, the expiratory phase, and the expiratory pause phase as determined through the motion data and determine at least one of an acoustic respiratory rate and a motion respiratory rate.

Another embodiment is directed to a method of monitoring breathing of a patient. The method includes: receiving information on the chest wall motion and sound that is separately sensed while the patient is breathing over a plurality of breath cycles; based on the chest wall motion, determining a start time and an end time for each of an inspiratory phase, an inspiratory pause phase, an expiratory phase, and an expiratory pause phase of each breath cycle of the patient; applying the start and end times for each of the phases of the breath cycles to the sound of the patient during the breath cycles; analyzing the sound of the patient exclusively at the expiratory pause phase and determining a noise floor; and determining actual patient sounds as the sounds that are received that are above the noise floor.

The method may include determining a noise floor threshold of the audio data as a multiple of a standard deviation.

Another embodiment is directed to a system configured to monitor breathing of a patient. The system includes a processing circuit configured to: receive information on the chest wall motion and sound that is separately sensed while the patient is breathing over a plurality of breath cycles; based on the chest wall motion, determine a start time and an end time for each of an inspiratory phase, an inspiratory pause phase, an expiratory phase, and an expiratory pause phase of each breath cycle of the patient; apply the start and end times for each of the phases of the breath cycles to the sound of the patient during the breath cycles; analyze the sound of the patient exclusively at the expiratory pause phase and determining a noise floor; and determine actual patient sounds as the sounds that are received that are above the noise floor.

The processing circuit may be configured to determine a noise floor threshold of the audio data as a multiple of a standard deviation.

Another embodiment is directed to a system to monitor breathing of a patient. The system includes: a first sensor configured to sense chest wall motion of the patient; a second sensor configured to concurrently sense breathing sound of the patient; a processing circuit configured to receive data from each of the first and second sensors; determine components of the breath cycle of the patient based on the chest wall motion, each of the breath cycles comprising an inspiratory phase, an inspiratory pause phase, an expiratory phase, and an expiratory pause phase; determine a beginning time and an ending time for each of the phases based on the chest wall motion; and determine at least one of an acoustic respiratory rate and a motion respiratory rate of the patient breathing base on the breathing sound during one or more of the inspiratory phase, the expiratory phase, and the expiratory pause phase as determined through the chest wall motion.

Another embodiment is directed to monitoring breathing of a patient that includes: sensing chest wall motion of the sound of the patient while breathing with the breathing including a plurality of breath cycles; based on the chest wall motion, determining a start time and an end time for each of an inspiratory phase, an inspiratory pause phase, an expiratory phase, and an expiratory pause phase of each breath cycle of the patient; applying the start and end times for each of the phases of the breath cycles to the sound of the patient during the breath cycles; analyzing the sound of the patient exclusively at the expiratory pause phase and determining a noise floor; and determining actual patient sounds as the sounds of the patient during the breath cycles that are above the noise floor.

The method may include determining a noise floor threshold of the audio data as a multiple of a standard deviation.

Another embodiment is directed to a system to monitoring breathing of a patient that includes: a first sensor to sense chest wall motion of the sound of the patient while breathing during breath cycles; a processing circuit configured to: based on the chest wall motion, determine a start time and an end time for each of an inspiratory phase, an inspiratory pause phase, an expiratory phase, and an expiratory pause phase of each breath cycle of the patient; apply the start and end times for each of the phases of the breath cycles to the sound of the patient during the breath cycles; analyze the sound of the patient exclusively at the expiratory pause phase and determining a noise floor; and determine actual patient sounds as the sounds of the patient during the breath cycles that are above the noise floor.

The processing circuit may be configured to determining a noise floor threshold of the audio data as a multiple of a standard deviation.

Another embodiment is directed to a method of monitoring breathing of a patient that includes: sensing motion of the patient and sound of the patient during a breath cycle; calculating an inspiratory pause phase of the breath cycle using the motion data including a starting time and an ending time; identifying the sound data that corresponds to the inspiratory pause phase of the breath cycle; and calculating a noise floor using the sound data during the expiratory pause phase.

Another embodiment is directed to a system for monitoring breathing of a patient. The system includes a processing circuit configured to: sense motion of the patient and sound of the patient during a breath cycle; calculate an inspiratory pause phase of the breath cycle using the motion data including a starting time and an ending time; identify the sound data that corresponds to the inspiratory pause phase of the breath cycle; and calculate a noise floor using the sound data during the expiratory pause phase.

Another embodiment is directed to a method of monitoring breathing of a patient that includes: sensing chest wall motion and sound of the patient during a breath cycle; calculating start and end times of an inspiratory phase and start and end times of an expiratory phase of the breath cycle based the motion data; calculating an average amplitude of the sound during the inspiratory phase of the breath cycle; calculating an average amplitude of the sound during the expiratory phase of the breath cycle; and calculating a comparison between the average amplitudes during the expiratory phase and the inspiratory phase.

The method may also include determining patient breathing based on a difference between the average amplitudes during the expiratory phase and the inspiratory phase.

The method may also include that calculating the comparison between the average amplitudes during the expiratory phase and the inspiratory phase includes dividing the average amplitude during the inspiratory phase by the average amplitude during the expiratory phase.

The method may include comparing the comparison to a threshold and determining that the patient is experiencing airway obstructive when the comparison is greater than the threshold.

The method may include calculating the average amplitude of the sound during the expiratory phase over a plurality of breath cycles.

The method may include calculating the average amplitude of the sound during the inspiratory phase over a plurality of breath cycles.

Another embodiment is directed to a system for monitoring breathing of a patient. The system includes a processing circuit configured to: sense chest wall motion and sound of the patient during a breath cycle; calculate start and end times of an inspiratory phase and start and end times of an expiratory phase of the breath cycle based the motion data; calculate an average amplitude of the sound during the inspiratory phase of the breath cycle; calculate an average amplitude of the sound during the expiratory phase of the breath cycle; and calculate a comparison between the average amplitudes during the expiratory phase and the inspiratory phase.

The processing circuit may be configured to determine patient breathing based on a difference between the average amplitudes during the expiratory phase and the inspiratory phase.

The processing circuit may be configured to calculate the comparison between the average amplitudes during the expiratory phase and the inspiratory phase comprises dividing the average amplitude during the inspiratory phase by the average amplitude during the expiratory phase.

The processing circuit may be configured to compare the comparison to a threshold and determining that the patient is experiencing airway obstructive when the comparison is greater than the threshold.

The processing circuit may be configured to calculate the average amplitude of the sound during the expiratory phase over a plurality of breath cycles.

The processing circuit may be configured to calculate the average amplitude of the sound during the inspiratory phase over a plurality of breath cycles.

Another embodiment is directed to a method of monitoring breathing of a patient that includes: sensing motion of the patient and sound of the patient during a breath cycle; calculating a first phase and a second phase of the breath cycle using the motion data; calculating an average amplitude of the sound during the first phase of the breath cycle with the start and end times of the first phase based on the motion data; calculating an average amplitude of the sound during the second phase of the breath cycle with the start and end times of the second phase determined based on the motion data; calculating a comparison between the average amplitudes during the first and second phases; and determining the patient breathing based on the comparison between the average amplitudes during the first and second phases.

The method may include that calculating the comparison between the average amplitudes during the first and second phases includes dividing the average amplitude during the first phase by the average amplitude during the second phase.

The method may include comparing the comparison to a threshold and determining that the patient is experiencing airway obstruction when the comparison is greater than the threshold.

The method may include calculating the average amplitude of the sound during the first phase over a plurality of breath cycles.

The method may include calculating the average amplitude of the sound during the second phase over a plurality of breath cycles.

Another embodiment is directed to a system for monitoring breathing of a patient. The system includes a processing circuit configured to: receive information on the motion of the patient and sound of the patient during a breath cycle; calculate a first phase and a second phase of the breath cycle using the motion data; calculate an average amplitude of the sound during the first phase of the breath cycle with the start and end times of the first phase based on the motion data; calculate an average amplitude of the sound during the second phase of the breath cycle with the start and end times of the second phase determined based on the motion data; calculate a comparison between the average amplitudes during the first and second phases; and determining the patient breathing based on the comparison between the average amplitudes during the first and second phases.

The system may include that calculating the comparison between the average amplitudes during the first and second phases includes the processing circuit dividing the average amplitude during the first phase by the average amplitude during the second phase.

The system may include that the processing circuit is configured to compare the comparison to a threshold and determining that the patient is experiencing airway obstruction when the comparison is greater than the threshold.

The method may include that the processing circuit is configured to calculate the average amplitude of the sound during the first phase over a plurality of breath cycles.

The system may include that the processing circuit is configured to calculate the average amplitude of the sound during the second phase over a plurality of breath cycles.

Another embodiment is directed to a method of monitoring breathing of a patient that includes: sensing motion of the patient and sound of the patient during a breath cycle; calculating an inspiratory phase and an expiratory phase of the breath cycle using the motion data; calculating an average amplitude of the sound during the inspiratory phase of the breath cycle with the start and end times of the inspiratory phase based on the motion data; calculating an average amplitude of the sound during the expiratory phase of the breath cycle with the start and end times of the expiratory phase determined based on the motion data; calculating a comparison between the average amplitudes during the inspiratory and expiratory phases; and determining the patient breathing based on the comparison between the average amplitudes during the inspiratory and expiratory phases.

Another embodiment is directed to a system to monitor breathing of a patient. The system includes: a processing circuit configured to receiving information on the motion of the patient and sound of the patient during a breath cycle; calculate an inspiratory phase and an expiratory phase of the breath cycle using the motion data; calculate an average amplitude of the sound during the inspiratory phase of the breath cycle with the start and end times of the inspiratory phase based on the motion data; calculate an average amplitude of the sound during the expiratory phase of the breath cycle with the start and end times of the expiratory phase determined based on the motion data; calculate a comparison between the average amplitudes during the inspiratory and expiratory phases; and determine the patient breathing based on the comparison between the average amplitudes during the inspiratory and expiratory phases.

One aspect is directed to using both acoustic and motion data to monitor the breathing. One embodiment includes a method of monitoring breathing of a patient. The method includes: concurrently sensing patient motion and breathing sounds during the breathing with the patient motion comprising an inclination of a sensor over time relative to two or more orthogonal axes; calculating breath cycles of the patient based on the patient motion with each of the breath cycles including an inspiratory phase, an inspiratory pause phase, an expiratory phase, and an expiratory pause phase with each of the phases having markers indicating a beginning of the phase and an ending of the phase; and using the sound data from at least one of the inspiratory phase, the expiratory phase, and the expiratory pause phase as determined through the motion data and determining at least one rate of the patient breathing.

Another aspect is directed to calculating an Inspiratory to Expiratory Acoustic Ratio (IEAR). One embodiment is directed to a method of monitoring breathing of a patient that includes sensing motion of the patient and sound of the patient during a breath cycle. The method includes: calculating an inspiratory phase and an expiratory phase of the breath cycle using the motion data; calculating an average amplitude of the sound during the inspiratory phase of the breath cycle with the start and end times of the inspiratory phase determined based on the motion data; calculating an average amplitude of the sound during the expiratory phase of the breath cycle with the start and end times of the expiratory phase determined based on the motion data; calculating a comparison between the average amplitudes during the expiratory phase and the inspiratory phase; and determining the patient breathing based on the relative difference.

The method may include that calculating the comparison between the average amplitudes during the expiratory phase and the inspiratory phase includes dividing the average amplitude during the inspiratory phase by the average amplitude during the expiratory phase.

The method may also include comparing the comparison to a threshold and determining that the patient is experiencing obstructive apnea when the comparison is greater than the threshold.

The method may also include calculating the average amplitude of the sound during the expiratory phase over a plurality of breath cycles.

The method may also include calculating the average amplitude of the sound during the inspiratory phase over a plurality of breath cycles.

Another embodiment is directed to a method of monitoring breathing of a patient. The method includes: sensing motion of the patient and sound of the patient during a breath cycle; calculating a first phase and a second phase of the breath cycle using the motion data; calculating an average amplitude of the sound during the first phase of the breath cycle with the start and end times of the first phase based on the motion data; calculating an average amplitude of the sound during the second phase of the breath cycle with the start and end times of the second phase determined based on the motion data; calculating a comparison between the average amplitudes during the first and second phases; and determining the patient breathing based on the comparison.

The method may include that calculating a comparison between the average amplitudes during the first and second phases includes dividing the average amplitude during the first phase by the average amplitude during the second phase.

The method may include that comparing the comparison to a threshold and determining that the patient is experiencing obstructive apnea when the comparison is greater than the threshold.

The method may include calculating the average amplitude of the sound during the first phase over a plurality of breath cycles.

The method may include calculating the average amplitude of the sound during the second phase over a plurality of breath cycles.

Another aspect is directed to calculating edge crossings on the motion plot to define the expiratory pause to get a better determination of noise floor

One embodiment is directed to a method of monitoring breathing of a patient. The method includes: sensing motion of the patient and sound of the patient during a breath cycle; calculating an inspiratory pause phase of the breath cycle using the motion data including a starting time and an ending time; identifying the sound data that corresponds to the inspiratory pause phase of the breath cycle; and calculating a noise floor using the sound data during the inspiratory pause phase.

The various aspects of the various embodiments may be used alone or in any combination, as is desired.

### BRIEF DESCRIPTION OF THE DRAWINGS

FIG. 1 is a schematic diagram of an acoustic sensor and a motion sensor operatively connected to a hub.

FIG. 2 is a schematic diagram of a hub.

FIG. 3 is a schematic diagram of a patient with motion and acoustic sensors attached and communicatively coupled to a hub that is in communication with a user device.

FIG. 4A is a display of motion data indicating an angle of a motion sensor over time.

FIG. 4B is a display of raw audio data indicating a frequency of sound over time with the breath cycles included based on motion data.

FIG. 4C is a display of audio data represented as an audio envelope over time with the breath cycles included based on motion data.

FIG. 4D is a display of audio data represented as amplitude of sound over time with a noise floor threshold based on the sound data during a pause of the breath cycle.

FIG. 5 is a flowchart diagram of a process to calculate RRm.

FIG. 6 is a flowchart diagram of a process to calculate a noise floor threshold.

FIG. 7 is a flowchart diagram of a process to calculate RRa.

FIG. 8 is a flowchart diagram of a process to calculate IEAR.

FIG. 9 is a flowchart diagram of a process to determine alarm triggers.

FIG. 10 is charts of motion data and acoustic data that includes an inspiratory pause phase.

### DETAILED DESCRIPTION

The present application is directed to a system for monitoring respiratory activity of a patient. The system includes a first sensor for sensing the acoustics of the patient during breathing and a second sensor for sensing chest wall motion of the patient during breathing. Based on the sensor data from the sensors, the system determines the patient's breathing. This may include whether the patient is experiencing regular breathing, slow breathing (hypopnea), fast breathing (tachypnea), no breathing (apnea), or obstructed breathing. The system may further trigger an alarm in predetermined situations.

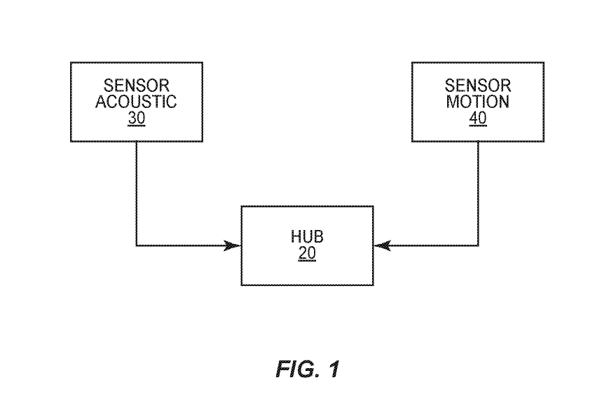

FIG. 1 illustrates a schematic depiction of the first and second sensors 30, 40 operatively connected to a hub 20. As illustrated in FIG. 1, the hub 20 may be physically positioned in proximity to the sensors 30, 40. The hub 20 may also be remotely located, such as at a server or other remote location. FIG. 1 includes the hub 20 in close proximity to the sensors 30, 40. In the various embodiments, communication between these elements may occur through various manners. These may include but are not limited to metallic wire, such as for example, twisted-pair wire or coaxial cable, fiber-optic cable, electromagnetic wireless channels, such as, for example, an IEEE 802.11 channel, a BLUETOOTH channel, a cellular channel, or an infrared channel, and/or any other appropriate type of wireline or wireless path for transferring information.

The first sensor 30 detects the breathing sounds made by the patient. Various types of sensors 30 may be used, including but not limited to electromagnetic induction, capacitance change, and piezoelectric sensors. In one specific embodiment, the sensor 30 is a bell/diaphragm configuration with an embedded microphone. The sensor 30 is configured to be attached to the patient in proximity to their trachea. The sensor 30 may be coupled directly to the patient (e.g., via adhesive, wrap, band), attached to the patient's clothing, or attached in proximity to the patient (e.g., on a bed adjacent to the patient).

The second sensor 40 is configured to detect motion of the patient during breathing. This may include coupling the sensor 40 to one or more of the patient's thorax, torso, chest, and ribcage region, each of which undergoes oscillatory movement during respiration. In one embodiment, the sensor 40 is positioned to detect motion of the chest wall of the patient. The sensor 40 may be coupled directly to the patient, or may be coupled to the patient's clothing.

One type of sensor is an orientation sensor 40 that determines the orientation measured as the inclination relative to two or more orthogonal axes against a known reference point, such as Earth, or Earth's gravity. The orientation sensor 40 may determine the orientation relative to X, Y, and Z axes. In one embodiment, the orientation sensor 40 is an accelerometer that measures the proper acceleration. Various other types of sensors 40 may also be used, including but are not limited to gyroscopes, magnetometers, analog and digital levels, and other various meters, levels, and switches.

The system may include one or more of each of the sensors 30, 40. The data from each of the sensors 30, 40 may be used to monitor the patient's breathing. In one embodiment, averages of each sensor type are used by the system. In another embodiment, the data from the different sensor types are compared to ensure each of the sensors are probably functioning. If the data from one of the sensors 30, 40 is outside of one or more predetermined thresholds, a signal may be sent for the sensor to be tested and/or replaced.

The sensors 30, 40 take measurements at a variety of different sampling rates. Preferably, the frequency is set that multiple measurements are captured during a single breath cycle of the patient. In one embodiment, the second sensor 40 has a sampling rate of between about 15-100 times per second.

One or both sensors 30, 40 may be configured to perform one or more calculations on the sensed data. The calculations are then transmitted to the hub 20 as necessary for the monitoring. Alternatively, one or both sensors 30, 40 may forward the data to the hub 20 which performs the various calculations.

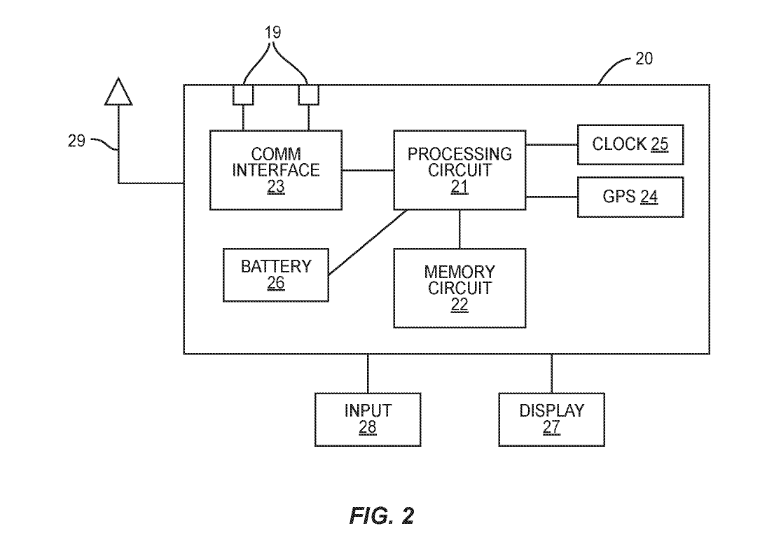

FIG. 2 schematically illustrates the hub 20 that includes a processing circuit 21, including, for example, one or more microprocessors, microcontrollers, Application Specific Integrated Circuits (ASICs) or the like, configured with appropriate software and/or firmware to control the overall operation of the system 20 according to program instructions stored in a memory circuit 22.

In one embodiment, the processing circuit 21 is configured to perform calculations to determine the acoustic and/or movement characteristics of the patient based on one or more signals received from the one or more sensors 30, 40. In other embodiments, the processing circuit 21 is configured to cause the raw sensor readings from the sensor 30 and/or sensor 40 to be transmitted to a remote location where the calculations are performed.

The hub 20 includes a computer-readable storage medium (shown as memory circuit 22), which stores instructions and/or data needed for operation. The memory circuit 22 may include both volatile and non-volatile memory, for example. A communication interface 23 may comprise a short-range wireless interface, such as a BLUETOOTH interface, RFID, ZIGBEE, or WIFI interface, a long range cellular phone or satellite communications interface, or one or more wired interfaces 19, such as a serial, USB, micro USB, FIREWIRE, Lightning, or Thunderbolt interface. There may be more than one communications interface 23. An antenna 29 may be configured for transmitting and receiving wireless signals to and from remote sources.

A GPS receiver 24 or other location detector is configured to determine location of the system. A clock 25 may be associated with the processing circuit 21 that measures the various timing requirements for specific events. The clock 25 may be independent from the processing circuit 21 as illustrated in FIG. 2, or may be incorporated within the processing circuit 21. The clock 25 may be configured to measure the specific time during each day, as well as to measure various time periods (i.e., days, weeks, months, years, etc.) An energy storage device 26 (e.g., a battery) is provided to power the various components.

The hub 20 may also include additional components. A display 27 may be configured to display information to the user and/or medical provider. The display 27 may comprise a liquid crystal display (LCD) or an organic light emitting diode (OLED) for example. Additionally, the display 27 may use printed electronic displays, electronic paper displays, or electronic ink technology to provide a thin, flexible and durable display to enable users to view information. An input 28 may provide for a user to enter applicable information. The input 28 may include a variety of formats, including but not limited to one or more buttons, touchpad, and keypad.

The hub 20 may also include or control one or more alerting devices for alerting the user of specified events or conditions. The alerting devices may comprise indicator lights that illuminate or generate lighting effects, speakers, beepers, buzzers, or other sound devices; and vibrators or other tactile devices. The alerting devices are controlled by the processing circuit 21 to notify the user when predetermined events or conditions occur. The alerts can be personalized and customized by the user to distinguish the alerts. The alerting devices may be included in the display 27 to include a message for the user.

The hub 20 is configured to receive sensor data from each sensor 30, 40. The hub 20 analyzes and compares the data relative to one or more predetermined thresholds. The hub 20 is configured to trigger an alarm in the event that one or more of the thresholds are exceeded. Continuously analyzing both breathing sound and chest wall motion provides for improved results over previous systems that analyze just one of these criteria.

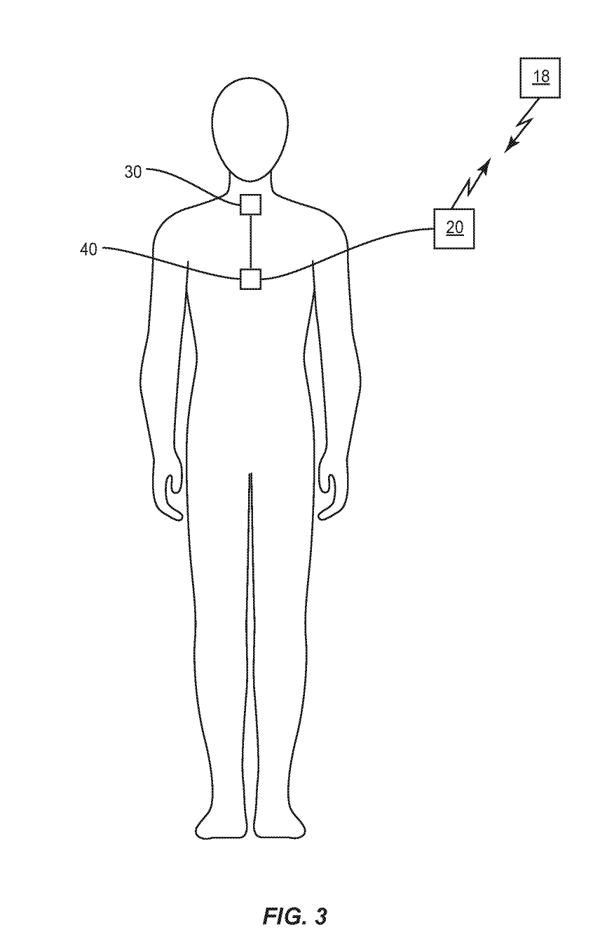

The hub 20 may be a stand-alone unit. As illustrated in FIG. 3, the hub 20 may be able to communicate with a user device 18, such as a mobile cellular telephone, a smart phone, a tablet, a laptop computer, and a personal digital assistant (PDA). The communication may be via wired or wireless path in a similar manner as those disclosed above.

In another embodiment, the functionality of the hub 20 is incorporated into the user device 18. The user device 18 is configured to run an Android or Apple iOS operating system. In another embodiment, the hub 20 and or user device 18 conveys the sensed data to a remote server that performs the various calculations on the data. In still other embodiments, calculations are performed by one or more hub 20, user device 18, and remote server.

The system monitors a patient's breathing using the combination of sensor data from the motion and sound sensors 30, 40. Using breathing sound analysis and chest wall motion analysis, the system detects the status of the patient. The motion and sound data are compared using this analysis to determine whether the patient is experiencing regular breathing, slow breathing (hypopnea), fast breathing (tachypnea), no breathing (apnea), or obstructed breathing. The system is configured to trigger an alarm if predetermined threshold parameters are exceeded for these criteria.

The use of both an acoustic sensor 30 and a motion sensor 40 provides for more accurate results. Acoustic data from sensor 30 and chest wall motion from sensor 40 are analyzed in parallel to increase the sensitivity and specificity for the different patient conditions. Sound and motion is compared in unison to more accurately determine the quality and quantity of breathing and reduce processing errors that may occur if just a single sensor type were used to monitor breathing.

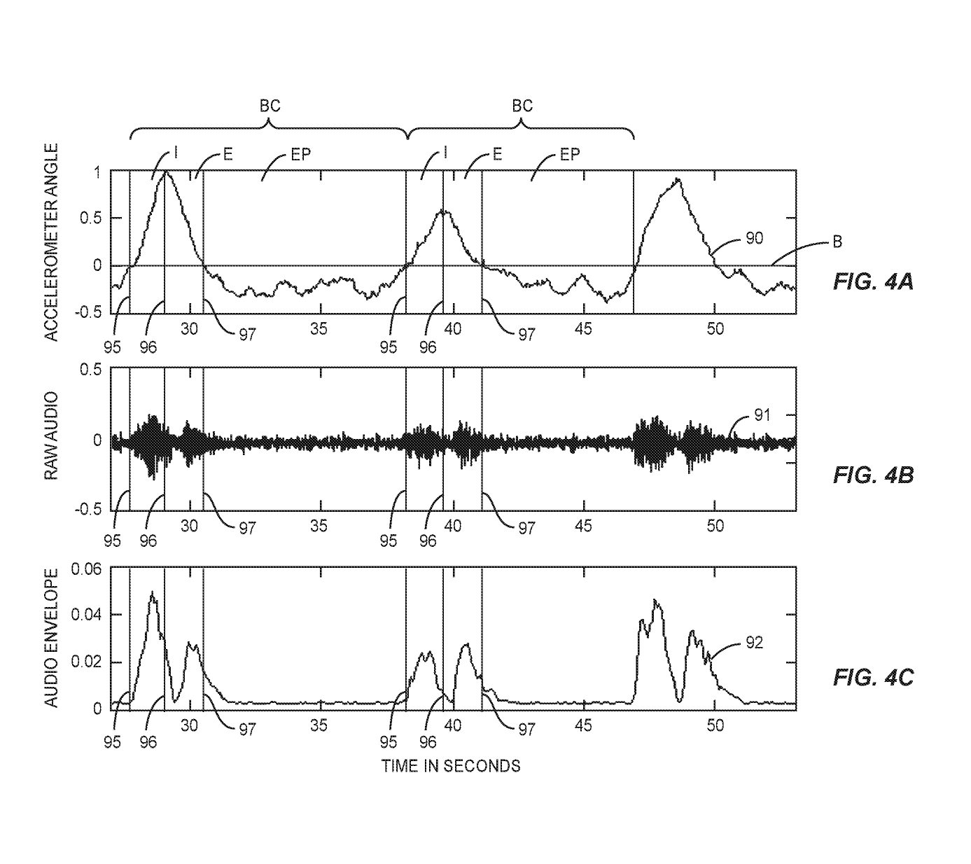

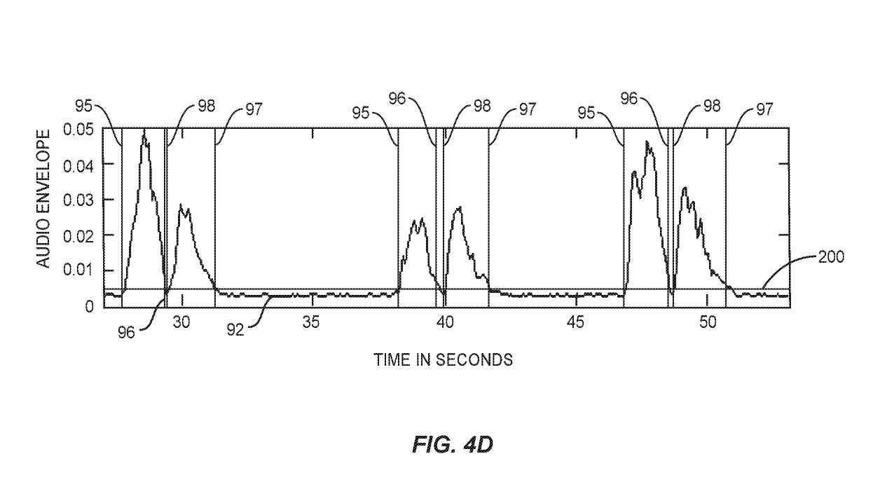

FIGS. 4A, 4B, and 4C illustrate embodiments of the outputs of the sensors 30, 40. The data in FIG. 4A illustrates accelerometer data 90 from the motion sensor 40. FIG. 4B illustrates raw audio data 91 from the sound sensor 30. FIG. 4C illustrates an audio envelope 92 of the processed raw audio data. For FIGS. 4B and 4C, observations regarding breath cycles BC of patient that are obtained from the motion data are applied to the sound data.

Each of the three data streams are aligned in time in FIGS. 4A, 4B, and 4C to illustrate the relationship between the sound data and the motion data. In one embodiment, the data from the sensors 30, 40 is received at the hub 20 in real time providing for a more straightforward time alignment. In other embodiments, one or both of the sound and motion data is manipulated to provide for the time alignment.

In one embodiment, the accelerometer data 90 is the change in angle of the motion sensor 40 over time. The motion sensor 40 is configured to detect movement over a number of different axes. A plane of action is determined by analyzing the motion and determining the plane in which a majority of the motion is occurring. Once the plane is determined, the motion is determined in first and second dimensions within the plane (e.g., motion along x and y axes). The motion data 90 is plotted as a change in degrees within the plane along one of the identified axes.

A baseline B is calculated for the motion data 90. In one embodiment, the baseline B is an average of the angle of the sensor 40 over a period of time. The baseline B is regularly calculated and may change based upon the changing data over time. In one embodiment as illustrated in FIG. 4A, the baseline B is set at zero and the plotted data is the sensed amounts reduced by the average.

FIG. 4A illustrates the motion data as the change in angle over time with the baseline B set at 0°. It is understood that the baseline B may be set at various levels. Further, different data may also be used for the plot 90 of the motion sensor 40. By way of example, the motion data may be a vector quantity of a magnitude and direction of the proper acceleration.

The acoustic sensor 30 is used to sense the sound of the patient's breathing. FIG. 4B illustrates the plot 91 of raw audio in frequency over time. This is the raw audio recorded during patient monitoring. This raw audio may be amplified one or more times as necessary.

Further acoustic data is illustrated in FIG. 4C as the audio envelope of the sensed sound. The audio envelope plot 92 is calculated by rectifiying (i.e., determining the absolute value of) the raw audio data 91. This rectified data is filtered with a low pass filter to obtain the audio envelope plot 92.

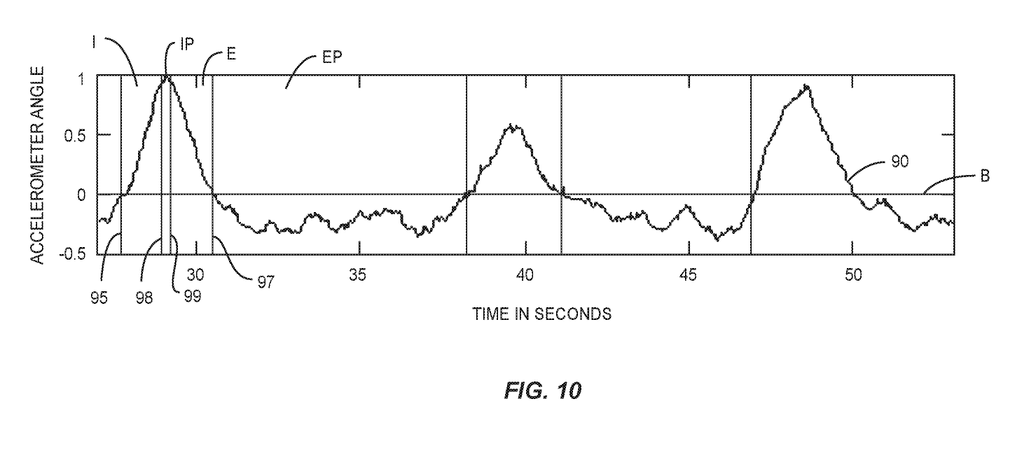

As shown in the motion data of FIG. 4A, a repeating breath cycle (BC) can be determined between adjacent points of the data. The BC of a patient includes an inspiratory (I) phase, an expiratory phase (E), and an expiratory pause phase (EP). The phases of the BC repeat with each breath. FIG. 10 illustrates the same data with greater delineation to include an inspiratory pause phase (IP). The inspiratory pause phase IP occurs between the inspiratory phase I and the expiratory phase E. The breath cycle BC of the motion data 90 of FIG. 4A is marked with the inspiratory phase I as the beginning of the breath cycle, although the breath cycle may also begin with any of the different phases.

The motion data 90 is evaluated to determine the phases of the breath cycle BC. The inspiratory phase I begins at the time when the motion data 90 passes through the baseline B in a positive direction. This point is illustrated by marker 95. The inspiratory phase I ends at the peak of the motion data 90 which is illustrated by marker 96. The expiratory phase E begins at the peak of motion (marker 96) and extends to the time when the motion data line 90 passes through the baseline B in a negative direction. This point is illustrated by marker 97. The expiratory pause phase (EP) extends from the end of the expiratory phase (marker 97) to the beginning of the inspiratory phase (marker 95) of the next breath cycle. As shown in the data, the breath cycle BC then repeats with the same phases. From viewing the data, each breath cycle BC includes the same phases. Each phase may vary in duration and/or intensity, depending upon the status of the patient. For example, the inspiratory phase I may be prolonged in patients with obstructive breathing.

As illustrated in FIG. 10 and stated above, the breath cycle BC may also include an inspiratory pause phase (IP). This inspiratory pause phase IP occurs between the inspiratory phase I and the expiratory phase E. In one embodiment, the IP is determined using data from the motion sensor 40 and is based on the peak of motion waveform. This may include the IP spanning a predefined amount of time before and after the peak as indicated by markers 98, 99. These amounts may be the same (i.e., the IP is centered about the peak) or may be different (i.e., the IP is not centered about the peak). The IP may also include just a predetermined period of time before the peak or a predetermined period of time after the peak.

As illustrated in the raw audio data 91 of FIG. 4B and the audio envelope data of FIG. 4C, the sound recorded during the inspiratory I and expiratory E phases register with highest sound levels. Further, the sound recorded during the expiratory pause phase EP register lesser sound levels.

The combination of the motion and sound data are used to monitor the patient's breathing. Using the combination of the two different inputs provides for more accurate results and reduces or eliminates false positive readings and other errors. Previous systems and methods have used just one type of data. The use of both types of data also allows for calculation of respiratory rates in the acoustic domain RRa and motion domain RRm, and an Inspiratory to Expiratory Acoustic Ratio (IEAR) which have each been found to be an effective manner of monitoring patient breathing. The use of both types of data also allows for using motion data to identify phases of the patient breath cycle. These phases can then be applied to the audio data to obtain a more accurate determination of a noise floor.

The different respiratory rates calculated using the motion data RRm and acoustic data RRa provide unique concepts in analyzing patient breathing. These rates are compared to one another and/or respiratory rate thresholds to determine the patient's respiratory status. Assessing the motion and sound data simultaneously reduces false positive readings, false negative readings, and other errors when monitoring for apnea.

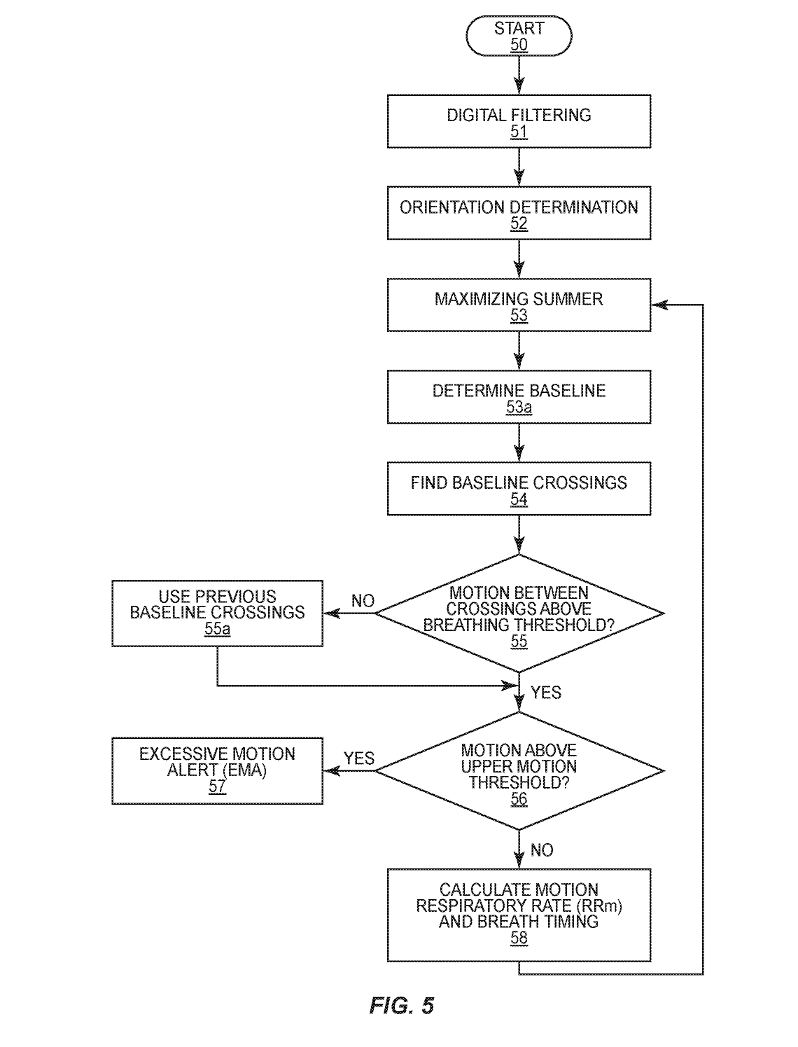

FIG. 5 illustrates the process of calculating the motion respiratory rate (RRm) using the motion data 90. The process starts (block 50) with digital filtering of the motion data (block 51). In one embodiment, this includes filtering the motion data with a low-pass filter. The digital filtering is performed on the three different planes of patient movement, for example, into movement in a first plane (e.g., x data), movement in a second plane (e.g., y data), and movement in a third plane (e.g., z data).

The orientation of the patient is determined by comparing acceleration due to gravity with the known orientation of the motion sensor 40 with respect to the patient. This orientation is then used to find the polarity of the three different planes (e.g., x, y, and z) (block 52). The motion data is maximized in a summer (block 53). This includes determining the two most correlated signals of the motion data, subtracting their time moving average, and summing the signals.

The baseline B of the motion data 90 (see FIG. 4A) is calculated (block 53a). In one embodiment, the baseline B is an average angle of the sensor 40 over a period of time. The baseline B is regularly calculated and changes per the data over time.

The process then calculates the zero crossing of the motion data (block 54). Using FIG. 4A as an example, this is the points of the motion plot 90 as it passes through the baseline B set at 0. The zero crossings provide a time period over which to calculate the RRm (block 54).

Using one period from a rising edge crossing (i.e., crossing the baseline in a positive direction) to a falling edge crossing (i.e., crossing the baseline in a negative direction), the maximum motion of the patient during this time period is calculated. Using the motion data 90 of FIG. 4A as an example, the rising edge crossing of the baseline B is indicated at marker 95 and the falling edge crossing is indicated at marker 97. This maximum motion during this time period corresponds to the inspiratory phase I and the expiratory phase E (and may also include the inspiratory pause phase IP (see FIG. 10).

This maximum motion of the patient motion during this time period is then compared to a lower motion threshold LMT (block 55). The LMT comparison determines if there is enough motion to be considered respiratory effort. If the patient motion during this time is below the LMT, this indicates that the motion was not enough to be analyzed as a breath and may be due to noise or shallow breathing. Since the motion does not indicate respiratory motion, the data is not considered during the timing analysis. Further, the previous data that are determined to be patient breathing are used, with the timing of when the data crosses the baseline used for timing purposes for the RRm (block 55a).

When the maximum motion during this time is above the LMT, the motion is then compared to an upper motion threshold UMT (block 56). The UMT is calculated from the previous body motions of the patient (e.g., the UMT is set as one or more standard deviations from a mean of previous body motions) and/or a predetermined threshold stored in memory. If the patient motion is above this UMT, the system determines that the patient is experiencing excessive motion and triggers an excessive motion alert (EMA) (block 57).

If the patient motion is below the UMT, the system calculates the RRm using the motion data. The RRm is respiratory rate for the patient over a period of time, such as over one minute. For example, the RRm may be the number of breath cycles completed within the period of time (e.g., 30 breath cycles per minute). The system also calculates the timing of the breath cycle BC. The timing of the breath cycle BC is the time for the patient to complete one breath cycle. This timing is calculated using two corresponding points on adjacent breath cycles (e.g., start of adjacent inspiratory phases, start of adjacent expiratory phases, etc.).

The motion of the patient may be calculated in a number of different manners. FIG. 4A illustrates the motion data as the change in angle over time with the baseline B set at 0°. In another embodiment, the motion data is a vector quantity of a magnitude and direction of the proper acceleration.

During the patient monitoring, the acoustic sensor 30 senses the patient's breathing in combination with ambient noise in the patient's environment. The sensed acoustic data includes a signal-to-noise ratio that includes the desired patient breathing and the unwanted ambient noise. A noise floor is calculated as a measure of the signal created from the sum of all the noise sources and unwanted signals with the noise defined as any signal other than the patient's breathing. The noise floor is determined using the sound data during the expiratory pause phase EP and/or inspiratory pause phase IP as defined by the motion data.

A drawback of previous systems using just one type of data is the inability to filter out ambient or background noises. Thus, it is not possible to obtain an accurate determination of the breath cycle BC. The present disclosure determines the noise floor during a period of relative small breathing sounds. The system determines a patient breath when the recorded audio data is above the noise floor. Thus, ambient noise that occurs in the patient's environment is not inadvertently counted as a breath.

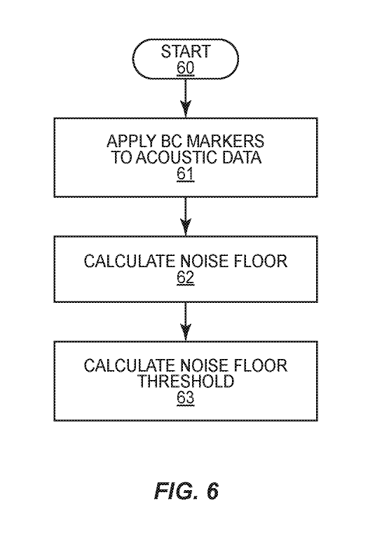

FIG. 6 illustrates the process for calculating the acoustic noise floor threshold. The process starts (block 60) and applies to the acoustic data 91, 92 the different phases of the breathing cycle BC that were determined using the motion data 90 (block 61). This is schematically illustrated in FIGS. 4B and 4C with the markers of the different phases being inserted along the acoustic data 91, 92 accordingly. As illustrated, lines 95, 96, 97 are used to differentiate the inspiratory phase I, expiratory phase E, and expiratory pause phase EP. It is understood that the markers may also be used to identify the inspiratory pause phase IP (see FIG. 10).

The acoustic noise floor is calculated using the sound data during one of the pauses EP, IP (block 62). This calculation may use one or both of the raw audio data 91 and the audio envelope 92. In one embodiment, the raw audio data during the EP is used to calculate the acoustic noise floor. This process determines that during the pauses EP, IP, the prevalent sound data is caused by ambient noise conditions. Therefore, using the sound data during this time allows for an accurate determination of the noise floor. Sounds that are above the noise floor are interpreted as breath sounds.

A noise floor threshold is determined by calculating the standard deviation of the audio data during one of the EP or IP (block 63). The noise floor threshold is a multiple of the standard deviation of the noise during the EP or IP. The noise floor threshold may also be calculated in other ways. This may include determining the standard deviation of the detected signal and setting the threshold between the amplitude of the standard deviation and the standard deviation of the noise floor using a decision theory principle.

FIG. 4D illustrates the audio envelope relative to the calculated noise floor threshold 200. As illustrated, the recorded sound data moves above and below the noise floor threshold 200 during various time periods. The system calculates the patient breathing from data that is above the noise floor threshold 200. FIG. 4D also includes the markers that are derived directly from the motion data. FIG. 4D also includes a marker 98 indicating an end of the inspiratory pause phase IP (i.e., the inspiratory pause phase extends for the time between markers 96 and 98).

The noise floor threshold is calculated at various times during the session. In one embodiment, this includes at the beginning of a patient session. The noise floor threshold may also be calculated periodically during the session to verify the noise floor threshold has remained constant.

In some embodiments, a preset noise floor threshold is used to monitor the patient. This preset noise floor threshold may be a default setting saved in the hub 20, or may be input by the user. However, it has been determined that the noise may vary under normal circumstances, and a preset threshold may not be accurate to meet desired sensitivity and specificity.

Calculating the noise floor threshold is necessary to differentiate specific phases of the patient breathing from the ambient noise. Noise data that does not rise above the noise floor threshold is not considered part of the inspiratory and expiratory phases I, E of the breath cycle. This noise is determined to be ambient/background noise of the patient's environment.

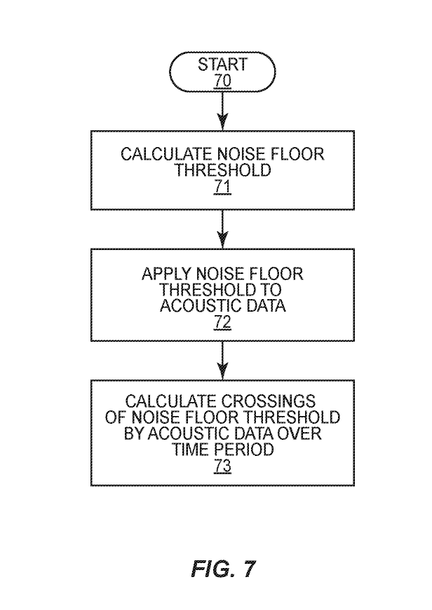

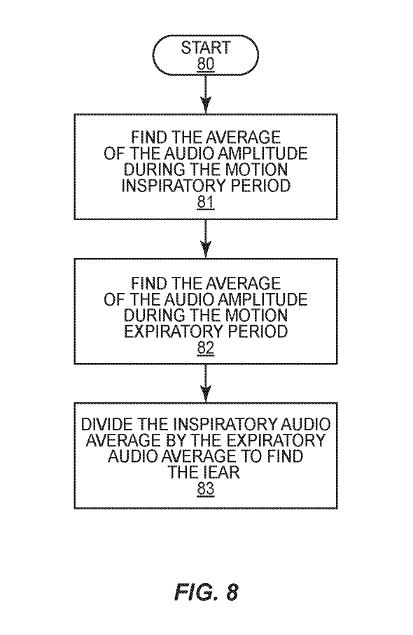

The system also calculates an acoustic respiratory rate (RRa) of the patient. This calculation is based on the acoustic data including one or both of the raw audio data 91 and audio envelope 92. The RRa is respiratory rate for the patient over a period of time, such as over one minute. FIG. 7 illustrates the process of finding the acoustic respiratory rate (RRa). The process starts (block 70) with the noise floor threshold being calculated from the acoustic data (either the raw audio or audio envelope) (block 71). The noise floor threshold is then applied to the acoustic data (block 72). FIG. 4D illustrates one example with the noise floor threshold 200 applied to the audio envelope. The edge crossings over a period of time are then calculated in which the plot 92 of the acoustic data moves through the noise floor threshold 200, both in a positive direction and a negative direction (block 73). Using FIG. 4D as an example, the acoustic data plot 92 moves through the noise floor threshold 200 in a positive direction at the beginning of both the inspiratory phase I and expiratory phase E and through the noise floor threshold 200 in a negative direction at the end of both the inspiratory phase I and expiratory phase E. This movement pattern continues for each breath cycle. In one embodiment, the RRa calculation determines the number of positive crossings and divides the number by two (2) to determine the number of breath cycles during the time period. In another embodiment, the RRa is calculated by determining the total number of noise floor threshold crossings (positive and negative) and dividing by four (4)(since there are four crossings per breath cycle).

The system further calculates an Inspiratory to Expiratory Acoustic Ratio IEAR. It has been determined that given the proper orientation of the motion sensor (40), the peak amplitudes of the motion waveform occur during the inspiratory phase I and the expiratory phase E. Therefore, the IEAR is calculated with the phases of the breath cycle BC determined with the motion data that are applied to the raw audio and/or audio envelope sound data. The IEAR analyzes differences in inspiratory and expiratory sounds to determine if there is an airway obstruction. Again, applying the boundaries of the breath cycle BC determined during the motion data analysis provides for more accurate results when analyzing the acoustic data 91, 92.

The process starts (block 80) and calculates an average amplitude of the audio data during the inspiratory phase I of the breath cycle BC (block 81). Schematically using the data from FIGS. 4B and 4C, this includes calculating the average of one or more of the raw audio data 91 and audio envelope 92 for the time period between markers 95 and 96. The process also calculates the average amplitude of the audio data 91, 92 during the expiratory phase E of the breath cycle BC (block 82). Again, using the data from FIGS. 4B and 4C, this includes the time period between markers 96 and 97. These averages of the different phases may be determined over two or more breath cycles BC.

The IEAR is calculated by dividing the average amplitude of the audio data during the inspiratory phase I by the average amplitude of the audio data during the expiratory phase E (block 83).

An advantage of the present methodology is the use of the motion data to determine the different phases of the breath cycle BC. The boundaries of the phases may then be applied to the sound data to more accurately analyze the sound data. When using an acoustic sensor alone to monitor breathing, ambient noise makes it difficult to accurately determine the different phases of the breath cycle BC. Identifying these sound characteristics is important in differentiating breathing sounds from ambient noise.

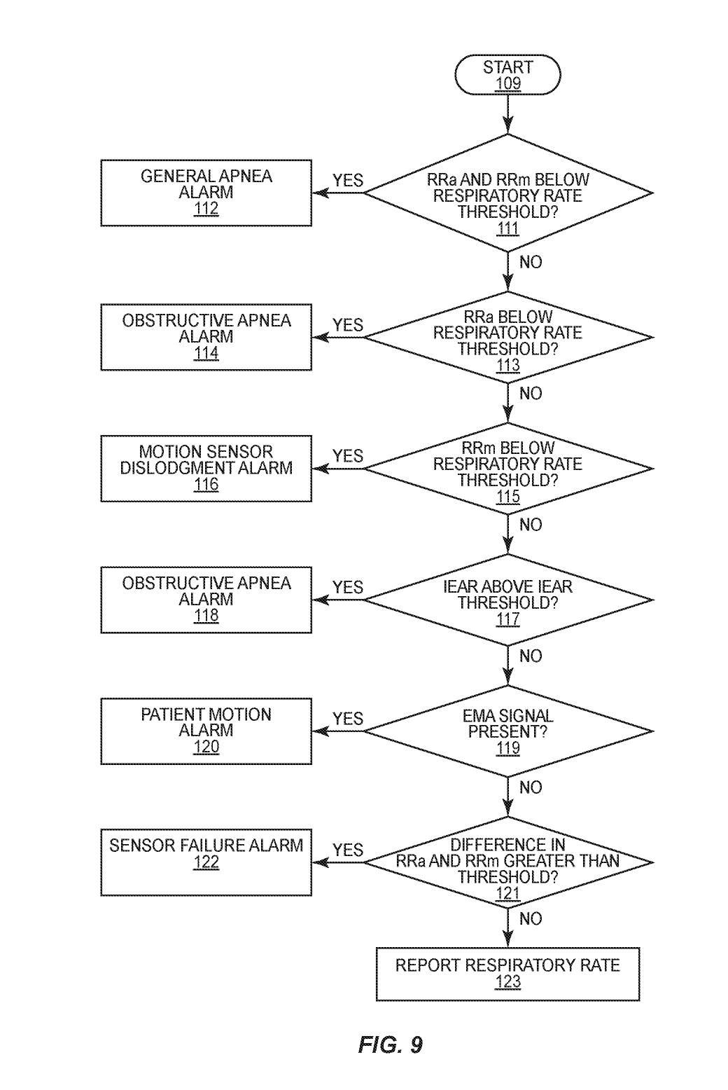

The system uses the various calculations to determine if there are concerns with the patient's breathing. FIG. 9 schematically illustrates a process of analyzing the patient's breathing. Although FIG. 9 schematically illustrates these processes performed in a particular order, it is understood that the process may be performed in various different orders of completion. Further, the process may include one or more of these calculations performed in separate processes.

As illustrated in FIG. 9, the process starts (block 109) and uses the calculated RRa, RRm, EMA, and IEAR. Both the RRa and RRm are compared to a minimum respiratory rate threshold (block 111). This threshold may be the same for both rates, or a different threshold may be used for each. If both rates RRa, RRm are below the threshold(s), a general apnea alarm is triggered (block 112).

If either respiratory rate RRa, RRm is above the threshold(s), the acoustic respiratory rate RRa is compared to a respiratory rate threshold (block 113). If the RRa is below its threshold, an alarm is triggered indicating that the patient is experiencing obstructve apnea (block 114).

If the RRa is not below its threshold, the motion respiratory rate RRm is compared to its respiratory rate threshold (block 115). This may be the same or different than the respiratory rate(s) used in blocks 111 and 113.

If the RRm is below its threshold, an alarm is triggered indicating that the motion sensor 40 may be dislodged from the patient (block 116).

If both the RRa and the RRm are above their threshold(s), the IEAR is then compared to an IEAR threshold (block 117). If the IEAR is above this threshold, an alarm is triggered indicating that the patient is experiencing obstructive apnea (block 118).

If the IEAR is below the IEAR threshold (block 117), the system checks if an excessive movement alert EMA was triggered by the motion algorithm (block 119) (see block 57—FIG. 5). If an EMA was triggered, then a patient motion alarm is triggered (block 120).

In one embodiment, the IEAR threshold is a predetermined value based on analysis of the breath sounds of patients experiencing obstructive apnea and snoring.

If an EMA signal is not present (block 119), the system determines an absolute difference between the acoustic respiratory rate RRa and the motion respiratory rate RRm. This difference is then compared to a threshold (block 121). If the difference is above this threshold, the system determines that the two respiratory rates RRa, RRm differ too much and triggers a sensor failure alarm (block 122). This alarm indicates that a sensor 30, 40 is poorly placed on the patient (block 122). If none of the alarms are triggered, the system calculates the respiratory rate as the average of the motion respiratory rate RRm and the acoustic respiratory rate RRa (block 123). The system is further able to calculate the patient's breathing rate based on one or more of the calculated values, such as the RRa and RRm. This may include determining whether the patient is experiencing regular breathing, slow breathing (hypopnea), or fast breathing (tachypnea).

In one embodiment, the threshold used for comparing with the difference between RRa and RRm (block 121) is a predetermined value determined from historical values and statistics.

The system may include the various calculations being performed at one or more locations. These calculations may be performed at one or more of the sensors 30, 40, the hub 20, a user device 18, and a remote server. In one embodiment, the calculations are each performed at the hub 20. In another embodiment, one or more calculations are performed at each of one or both sensors 30, 40, the hub 20, and a remote server.

In the various processes, alarms may be triggered indicating a particular situation (e.g., general apnea alarm, obstructive apnea alarm, sensor failure alarm). The alarms may be sent to one or more of medical personnel, emergency services contact, the user device 18, the patient's device, a caregiver, and others. In one embodiment, the hub 20 includes one or more alerting devices that are activated during an alarm. The alerting devices may include indicator lights that illuminate or generate lighting effects, speakers, beepers, buzzers, or other sound devices, and vibrators or other tactile devices. The alerting devices are controlled by the processing circuit 21 to notify the user when predetermined events or conditions occur. The alerts can be personalized and customized by the user to distinguish the alerts. The alerting devices may be included in the display 27 to include a message for the user and/or medical personnel. The alerts may also be sent to a nursing station when the system is used in a hospital environment.

The triggered alarms may also be classified into different levels of degree. A first type of alarm may indicate an abnormality with the patient or system (e.g., motion sensor dislodgment alarm, patient motion alarm). This first type of alarm may result in the hub 20 performing additional operations or a higher frequency of operations. This may include but is not limited to performing a higher testing rate for sound and/or motion data, performing one or more additional testing algorithms on the patient using the existing data or additional data either previously detected or to be detected. Additionally, the data resulting in the triggered alarm may be flagged in a manner and presented to medical personnel. This level of triggered alarm may also indicate benign behaviors that are detected such as excessive motion from the patient indicating physical motion not consistent with a resting patient.

A second type of alarm may occur during a more serious detected occurrence (e.g., general apnea alarm, obstructive apnea alarm). This may include but is not limited to sending a signal for immediate medical personnel, sounding an audible alarm, increased data tracking frequency, additional data tracking, and others.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A method of monitoring breathing of a patient, the method comprising:
receiving chest wall motion and breath sounds that are sensed concurrently while the patient is breathing;
determining components of the breath cycle of the patient based on the chest wall motion, each of the breath cycles comprising an inspiratory phase, an inspiratory pause phase, an expiratory phase, and an expiratory pause phase;
determining a beginning time and an ending time for each of the phases based on the chest wall motion; and
determining at least one of an acoustic respiratory rate and a motion respiratory rate of the patient based on the breath sounds during one or more of the inspiratory phase, the expiratory phase, and the expiratory pause phase as determined through the chest wall motion.

2. The method of claim 1, further comprising establishing a baseline for the chest wall motion and determining for each breath cycle that the inspiratory phase begins when the motion data passes through the baseline in a positive direction and the expiratory phase ends when the motion data passes through the baseline in a negative direction.

3. The method of claim 1, further comprising determining that the expiratory pause phase occurs between an end of the expiratory phase in a first breath cycle and a start of the inspiratory phase in a second breath cycle.

4. The method of claim 3, further comprising determining that the chest wall motion remains below the baseline during the entirety of the expiratory pause phase.

5. The method of claim 2, wherein establishing the baseline for the chest wall motion comprises calculating an average inclination of the sensor over a period of time.

6. The method of claim 2, further comprising determining a motion respiratory rate based on the chest wall motion for the breath cycles that occur over a period of time using the chest wall motion only for the breath cycles that have a maximum chest wall motion that is above a predetermined threshold.

7. The method of claim 1, further comprising:
calculating a noise floor threshold based on the breath sounds during the expiratory pause phase of a plurality of the breath cycles;
applying the noise floor threshold to the breath sounds;
calculating a number of edge crossings at which the breath sounds crosses over the noise floor threshold;
determining an acoustic respiratory rate based on the number of edge crossings that occur within a predetermined time period.

8. The method of claim 1, wherein receiving chest wall motion and breath sounds that are sensed concurrently while the patient is breathing comprises receiving the chest wall motion from a first sensor that is attached to the patient and receiving the breath sounds from a separate second sensor that is attached to the patient.

9. The method of claim 1, further comprising wirelessly receiving the chest wall motion and the breath sounds from sensors that are attached to the patient.

10. A method of monitoring breathing of a patient, the method comprising:
sensing chest wall motion comprising an inclination of a sensor over time relative to two or more orthogonal axes;
while sensing the chest wall motion, also sensing breath sounds of the patient;
calculating breath cycles of the patient based just on the patient motion with each of the breath cycles comprising an inspiratory phase, an inspiratory pause phase, an expiratory phase, and an expiratory pause phase, each of the phases having a start and an end;
using the breath sounds data from at least one of the inspiratory phase, the expiratory phase, and the expiratory pause phase as determined through the motion data and determining at least one of an acoustic respiratory rate and a motion respiratory rate.

11. The method of claim 10, further comprising establishing a baseline for the chest wall motion and determining that the inspiratory phase begins for each breath cycle when the motion data passes through the baseline in a positive direction.

12. The method of claim 11, further comprising determining that the expiratory phase ends when the motion data passes through the baseline in a negative direction.

13. The method of claim 10, further comprising determining that the expiratory pause phase occurs between an end of the expiratory phase in a first breath cycle and a start of the inspiratory phase in a second breath cycle.

14. The method of claim 11, further comprising determining that the motion data remains below the baseline during the entirety of the expiratory pause phase.

15. The method of claim 11, wherein establishing the baseline for the chest wall motion comprises calculating an average angle over a period of time.

16. The method of claim 10, further comprising determining whether a maximum chest wall motion during the inspiratory phase and the expiratory phase is above an upper motion threshold.

17. The method of claim 10, further comprising determining a motion respiratory rate based on the chest wall motion for the breath cycles that occur over a period of time using the chest wall motion only for the breath cycles that have a maximum chest wall motion that is above a predetermined threshold.

18. A method of monitoring breathing of a patient, the method comprising:
receiving information on the chest wall motion and sounds that are concurrently sensed while the patient is breathing over a plurality of breath cycles;
based on the chest wall motion, determining a start time and an end time for each of an inspiratory phase, an inspiratory pause phase, an expiratory phase, and an expiratory pause phase of each breath cycle of the patient;
applying the start and end times for each of the phases of the breath cycles to the sounds of the patient during the breath cycles;
analyzing the sounds of the patient exclusively at the expiratory pause phase and determining a noise floor; and
determining actual patient sounds as the sounds that are received that are above the noise floor.

19. The method of claim 18, further comprising wirelessly receiving the information on the chest wall motion and sounds from sensors that are attached to the patient.
